(19) United States
(12) Patent Application Publication
(10) Pub. No.: US 2024/0341926 A1
(43) Pub. Date: Oct. 17, 2024

(54) SYSTEM FOR VISUALIZING AT LEAST ONE THREE-DIMENSIONAL VIRTUAL MODEL OF AT LEAST PART OF A DENTITION

(71) Applicant: Hirsch Dynamics Holding AG, Wollerau (CH)

(72) Inventor: Markus HIRSCH, Wollerau (CH)

(73) Assignee: Hirsch Dynamics Holding AG, Wollerau (CH)

(21) Appl. No.: 18/749,693

(22) Filed: Jun. 21, 2024

Related U.S. Application Data
(63) Continuation of application No. PCT/EP2021/087537, filed on Dec. 23, 2021.

(51) Int. Cl. A61C 9/00; G06F 3/04815; G06T 17/00

(52) U.S. Cl. CPC A61C 9/0053; G06F 3/04815; G06T 17/00

(57) ABSTRACT

A system for visualizing at least one three-dimensional virtual model of at least part of a dentition has at least one screen and least one processor configured to generate an on-screen layout and having a plurality of graphical control elements. The processor is configured such that operator interaction leads to the invocation of an associated function. The processor has a user interface with a viewer region for visualizing a three-dimensional virtual model of at least part of a dentition, and at least a first and a second category of individual graphical control elements. Elements of the first category are hidden and/or spatially separated from and/or differently colored compared to individual graphical controls of the second category such that accidental interaction with elements from the first category is reduced. The processor indicates availability of associated functions by a color of the function name and/or a color of each graphical control element.

### CROSS REFERENCE TO RELATED APPLICATIONS

This application is a continuation of and Applicant claims priority under 35 U.S.C. § 120 of International Application No. PCT/EP2021/087537 filed on Dec. 23, 2021, The international application under PCT article 21(2) was published in English. The disclosure of the aforesaid International Application is hereby incorporated by reference.

### BACKGROUND OF THE INVENTION

The present invention relates to the field of orthodontics and, more specifically, to an application software-based method for visualizing a three-dimensional virtual model of at least part of a dentition and comprising an on-screen layout in which an operator has access to the virtual model and a plurality of graphical control elements whereby an operator interaction with each individual graphical control element may be undertaken either by touchscreen or by means of an external pointing device and in which said operator interaction leads to the invocation of an associated function.

The ability to digitally visualize and manipulate a dentition as part of an orthodontic treatment plan allows for improved conveyance of information between dental practitioners, patients and corrective appliance fabricators and a more structured and robust method of treatment planning. A software-based method for visualizing at least part of a dentition is already known from the state of the art. In U.S. Pat. No. 8,260,591 B2, a software system comprising a view and dynamic content is provided, whereby rendering the view is based on the dynamic content for the purpose of generating a treatment in an efficient and cost-effective manner.

A disadvantage of the prior art is that elements of the software functionality may be unintentionally activated through inadvertent operator interaction with the graphical control elements composite in the user interface. Moreover, it is often the case that new operators require a significant amount of time before becoming proficient in the operation of such systems and the possibility to implement incorrect procedures is not insignificant.

### SUMMARY OF THE INVENTION

It is an object of the present invention to provide an application software-based method for visualizing at least one three-dimensional virtual model of at least part of a dentition within a viewer region and a plurality of graphical control elements which is appropriate to eliminate at least some of the disadvantages of the prior art.

The object of the present invention is accomplished by a system for visualizing at least one three-dimensional virtual model of at least part of a dentition, comprising:
at least one screen
at least one processor configured to generate an on-screen layout on said screen, said on-screen layout giving access to an operator to said virtual model and having a plurality of graphical control elements whereby the processor is configured to provide for an operator interaction with each of said plurality of graphical control elements, preferably by touchscreen or by means of an external pointing device, and wherein said processor is configured such that said operator interaction leads to the invocation of an associated function.

The at least one processor is configured to generate said on-screen layout having:
a user interface comprising:
   a viewer region wherein a three-dimensional virtual model of at least part of a dentition may be visualized,
   at least a first and a second category of individual graphical control elements, from the plurality of graphical control elements, wherein elements of the first category of the two different categories are hidden and/or spatially separated from and/or differently colored compared to individual graphical controls of the second category such that accidental interaction with said individual graphical control elements from said first category is reduced,
the processor being configured to indicate availability of associated functions by a color of the function name and/or a color of each of the plurality of graphical control elements.

Individual graphical control elements, from said first category of graphical control elements represent associated functions of heightened importance and/or criticality.

The at least one graphical control element from the plurality of graphical control elements may be positioned adjacent to a related function name. The identification of the at least one graphical control element with the invocation of an associated function may therefore be enhanced such that the invocation of an undesired function is minimized.

At least one of the plurality of graphical control elements may be in the form of a dialogue box.

The processor may be configured to measure a potentially imminent operator interaction with an individual graphical control element from the plurality of graphical control elements and to activate a signal upon approaching a threshold value and deactivate said signal upon retreating from said threshold value based on a measured potentially imminent operator interaction with an individual graphical control element from said plurality of graphical control elements. The threshold value may be in the form of a distance and/or a pressure.

The processor may be configured to change the color of each of the plurality of graphical control elements following an operator interaction. An operator interaction may change the color from a first color to a second color following a first operator interaction and may change the color from a second color to a first color following a second operator interaction.

The user interface comprising a viewer region wherein a three-dimensional virtual model of at least part of a dentition may be visualized, the processor may be configured to, within the viewer region, visualize the three-dimensional virtual model of at least part of a dentition in a translationally and/or rotationally displaced state.

The user interface comprising a plurality of graphical control elements whereby a potentially imminent operator interaction with an individual graphical control element from the plurality of graphical control elements may be a measurable quantity whereby the measurable quantity may be measured in terms of either the proximity of an external element or the pressure applied by an external element, in the case operator interaction is undertaken by touchscreen, or in terms of the proximity of a mouse cursor element in the case operator interaction is undertaken by means of an external pointing device. Provision may therefore be made for a plurality of different input methods, e. g. touchscreen, stylus, mouse, trackpad, keyboard, on-screen keyboard etc.

The user interface comprising a plurality of graphical control elements whereby a measured potentially imminent operator interaction with an individual graphical control element from the plurality of graphical control elements may activate a signal upon approaching a threshold value and may deactivate the signal upon retreating from the threshold value, whereby, the signal may be in the form of a blue glowing effect whereby the effect may be spatially delimited to the immediate region of the individual graphical control element. The signal may also be in the form of pulsating glowing effect. Preferably the signal may be delimited to a region within 10 mm of the individual graphical control element.

The user interface comprising a plurality of graphical control elements whereby the color of each of the plurality of graphical control elements may change following an operator interaction, whereby the color of each of the plurality of graphical control elements may change from black to blue, in the event an associated function may be activated and/or the color of each of the plurality of graphical control elements may change from blue to black, in the event an associated function may be deactivated.

The user interface may comprise a layout of a plurality of graphical control elements whereby a plurality of subdivisions of the plurality of graphical control elements may be grouped according to the associated functions invoked following a user interaction with each individual graphical control element from the plurality of graphical control elements whereby the groupings may comprise manipulation of the visualized dentition, placement and manipulation of object elements, analysis tools, modification methods, administrative functions and/or further critical functions etc. whereby the individual groupings may be further demarcated by the presence of associated headings and/or lines and/or shaded regions. The groupings may not be limited to those here presented and provision may be made for groupings to be added, removed and/or differently arranged.

The processor may be configured to hide individual graphical control elements, from the plurality of graphical control elements, representing associated functions of heightened importance and/or criticality, from the remainder of a plurality of graphical control elements such that accidental interaction with the hidden graphical control elements is minimized whereby access to the hidden graphical control elements may be achieved through operator interaction with a spatially separated and/or differently colored graphical control element. Examples of such functions may include any which result in permanent and/or irreversible change to the treatment plan and/or confirmation of a selected action to an external stakeholder etc.

The processor may be configured to visualize at least one of the spatially separated graphical control elements translationally displaced by a distance exceeding the typical separation distance between the remainder of the graphical control elements.

The processor may be configured to accept an operator interaction within the dialogue box only upon the input of a preset minimum number of characters into the dialogue box. Preferably acceptance of an operator interaction may be possible only after the input of a minimum of 10 characters into the dialogue box.

### BRIEF DESCRIPTION OF THE DRAWINGS

Further details and advantages of the present invention are explained in more detail below with reference to the description of the figures and with reference to the exemplary embodiments illustrated in the drawings. In the drawings, the figures show:

FIG. 1 shows an application software-based method for visualizing at least one part of a dentition,

FIG. 2 shows a first representation of a user interface of an application software-based method for visualizing at least one part of a dentition indicating individual groupings of graphical control elements,

FIG. 3 shows a second representation of a user interface of an application software-based method for visualizing at least one part of a dentition indicating the expansion of a group of graphical control elements,

FIG. 4 shows a third representation of a user interface of an application software-based method for visualizing at least one part of a dentition indicating spatially separated groupings of graphical control elements, and

FIG. 5 shows a graphical control element in the form of a dialogue box, normally hidden from view and accessible only through an operator interaction with a spatially separated graphical control element.

### DETAILED DESCRIPTION OF THE EMBODIMENTS

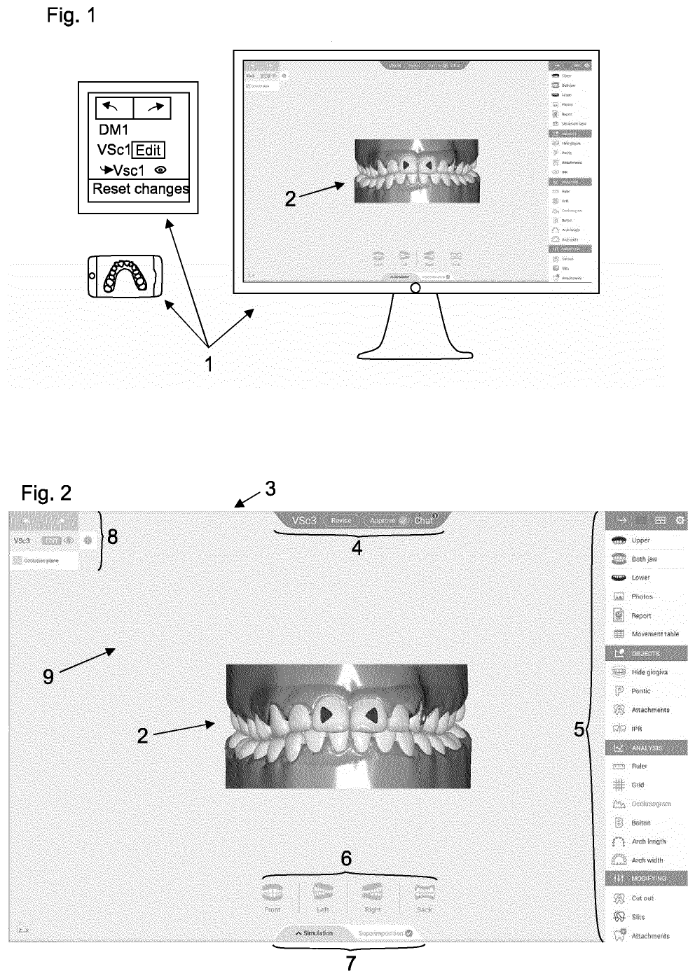

FIG. 1 discloses an application software 1 based method for visualizing a three-dimensional virtual model 2 of at least part of a dentition. The application software 1 based method is independent of the platform upon which the software is running and the operator interaction methods available to the platform. In a first embodiment, an operator may interact with the application software 1 based method by touch and/or through use of a dedicated pointing device in the case of touchscreen operation. In a second embodiment an operator may interact with the application software 1 based method through use of an external pointing device controlling an on-screen pointer in the case of non-touchscreen operation.

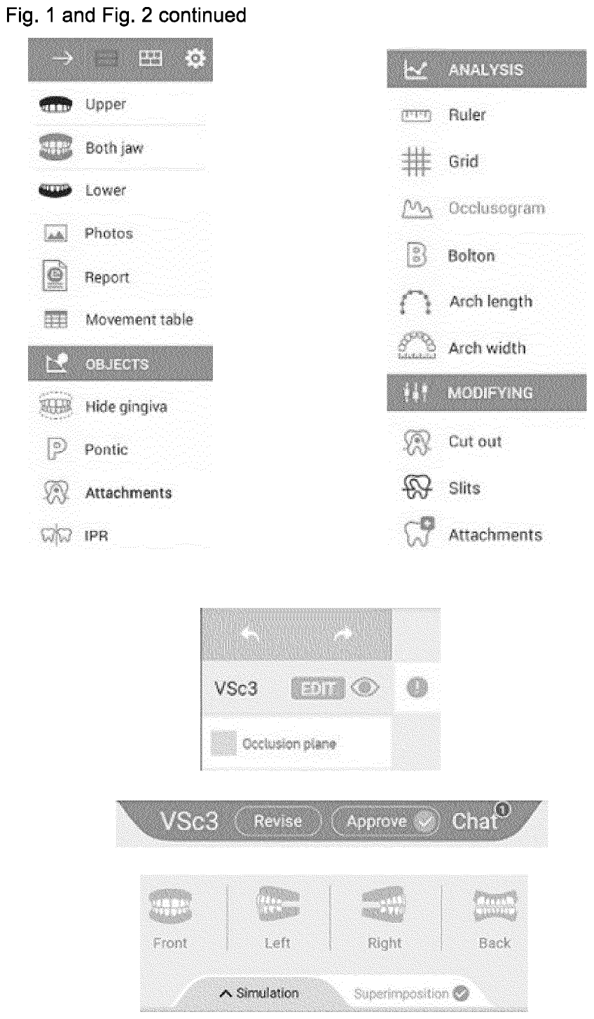

FIG. 2 discloses an on-screen layout 3 of an application software 1 based method for visualizing a three-dimensional virtual model 2 of at least part of a dentition whereby the three-dimensional virtual model 2 is translationally and rotationally displaceable within a viewer region 9. A plurality of graphical control elements 4, 5, 6, 7, 8 are arranged around the periphery of the viewer region 9 whereby the plurality of graphical control elements 4, 5, 6, 7, 8 are grouped according to the associated functions invoked following a user interaction with each individual graphical control element from the plurality of graphical control elements 4, 5, 6, 7, 8 whereby the groupings comprise manipulation of the visualized dentition, placement and manipulation of object elements, analysis tools, modification methods, administrative functions, further critical functions etc.

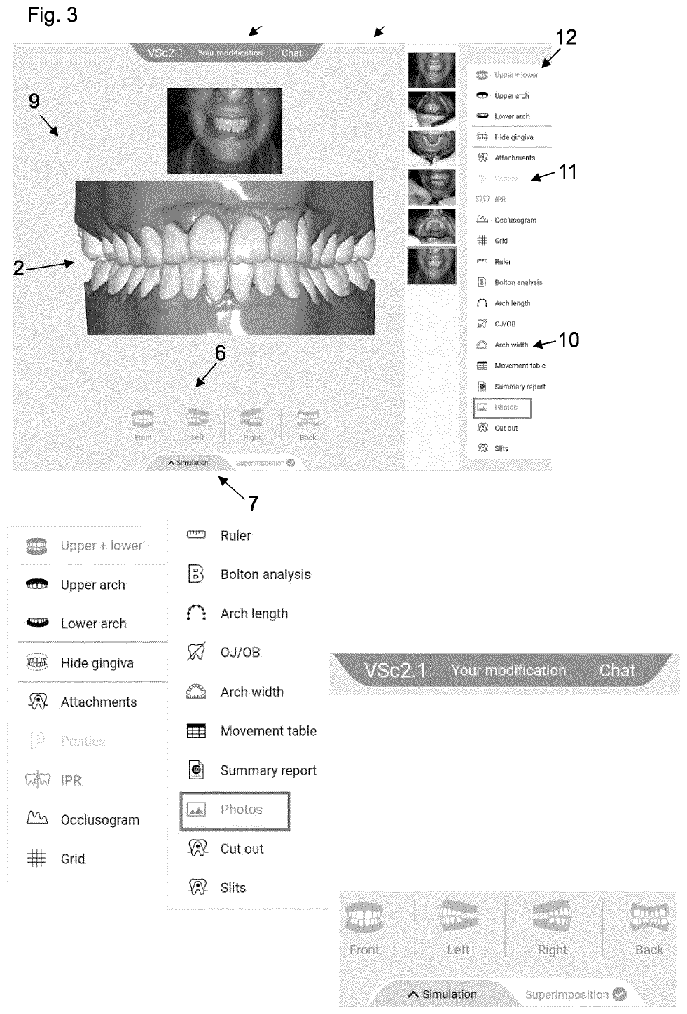

FIG. 3 discloses an on-screen layout 3 of an application software 1 based method for visualizing a three-dimensional virtual model 2 of at least part of a dentition comprising a plurality of graphical control elements 4, 5, 6, 7, 8 whereby the color of each of the plurality of graphical control elements 4, 5, 6, 7, 8 indicates the availability of associated functions, whereby the color, in the case an associated function is available, is black 10 and/or the color, in the case an associated function is unavailable, is gray 11. Furthermore, the color of each of the plurality of graphical control elements 4, 5, 6, 7, 8 changes following an operator interaction, whereby the color of each of the plurality of graphical control elements 4, 5, 6, 7, 8 changes from black 10 to blue 12, in the event an associated function is activated and/or the color of each of the plurality of graphical control elements 4, 5, 6, 7, 8 changes from blue 12 to black 10, in the event an associated function is deactivated.

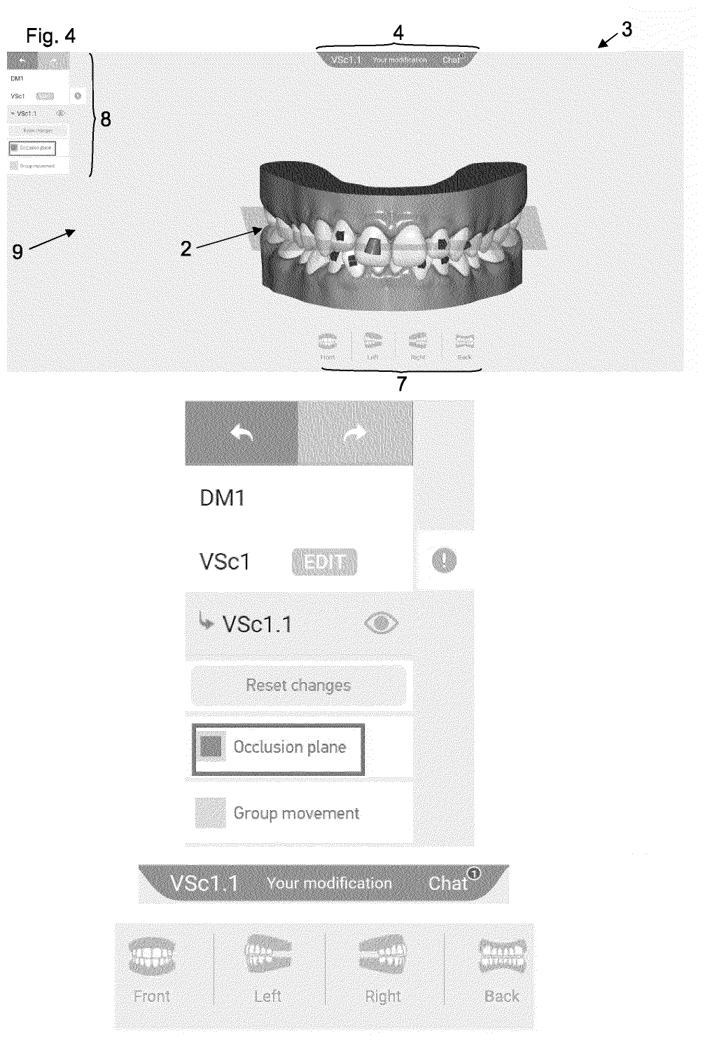

FIG. 4 discloses an on-screen layout 3 of an application software 1 based method for visualizing a three-dimensional virtual model 2 of at least part of a dentition comprising a plurality of graphical control elements 4, 5, 6, 7, 8 whereby individual graphical control elements, from the plurality of graphical control elements 4, 5, 6, 7, 8, belonging to a first category (representing associated functions of heightened importance and/or criticality), are spatially separated and/or differently colored from the remainder of a plurality of graphical control elements 4, 5, 6, 7, 8 (belonging to a second category) such that accidental interaction with the spatially separated graphical control elements 4, 5, 6, 7, 8 is minimized whereby at least one of the spatially separated graphical control elements 4, 5, 6, 7, 8 are translationally displaced by a distance at least approximating the height of the user interface 4, 8.

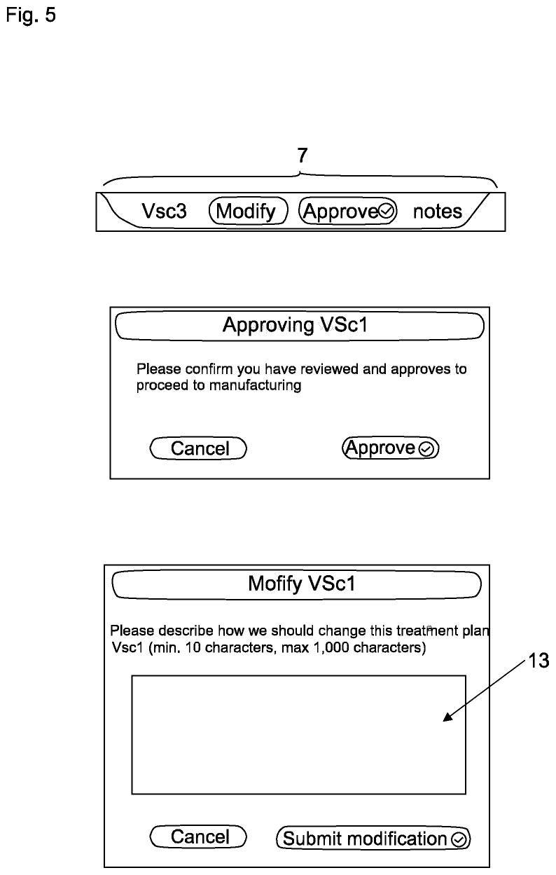

FIG. 5 discloses a spatially separated graphical control element 7 from a plurality of graphical control elements 4, 5, 6, 7, 8, belonging to the second category, whereby an operator interaction with the spatially separated graphical control element 7 opens a further graphical control element in the form of a dialogue box 13 (belonging to the first category) whereby acceptance and/or submission of an operator interaction with the dialogue box 13 is only possible upon the input of at least 10 characters into the dialogue box 13. Under normal operating conditions, the dialogue box 13 is hidden from view and is only accessible through an operator interaction with a spatially separated graphical control element 7.

What is claimed is:

1. A system (1) for visualizing at least one three-dimensional virtual model (2) of at least part of a dentition, comprising:
at least one screen,
at least one processor configured to generate an on-screen layout (3) on said screen, said on-screen layout (3) giving access to an operator to said virtual model (2) and having a plurality of graphical control elements (4, 5, 6, 7, 8), wherein the processor is configured to provide for an operator interaction with each of said plurality of graphical control elements (4, 5, 6, 7, 8), wherein said processor is configured such that said operator interaction leads to the invocation of an associated function,
wherein at least one said processor is configured to generate said on-screen layout (3) having:
a user interface comprising:
a viewer region (9) wherein a three-dimensional virtual model (2) of at least part of a dentition may be visualized,
at least a first and a second category of individual graphical control elements, from said plurality of graphical control elements, wherein elements of said first category of the two different categories are hidden and/or spatially separated from and/or differently colored compared to individual graphical controls of said second category such that accidental interaction with said individual graphical control elements from said first category is reduced,
wherein said processor is configured to indicate availability of associated functions by a color of said function name and/or a color of each of said plurality of graphical control elements (4, 5, 6, 7, 8).

1. The system (1) according to claim 1, wherein said user interface comprises a viewer region (9), wherein a three-dimensional virtual model (2) of at least part of a dentition may be visualized in said viewer region, said processor being configured to, within said viewer region (9), visualize said three-dimensional virtual model (2) of at least part of a dentition in a translationally and/or rotationally displaced state.

2. The system (1) according to claim 1, wherein said user interface comprises a plurality of graphical control elements (4, 5, 6, 7, 8), wherein at least one graphical control element of said plurality of graphical control elements (4, 5, 6, 7, 8) is positioned adjacent to a related function name.

3. The system (1) according to claim 1, wherein said user interface comprises a layout (3) of a plurality of graphical control elements (4, 5, 6, 7, 8), wherein a plurality of subdivisions of said plurality of graphical control elements (4, 5, 6, 7, 8) are grouped according to the associated functions invoked following a user interaction with each individual graphical control element from said plurality of graphical control elements (4, 5, 6, 7, 8), wherein said groupings comprise manipulation of the visualized dentition, placement and manipulation of object elements, analysis tools, modification methods, administrative functions and/or further critical functions, wherein said individual groupings are further demarcated by the presence of associated headings and/or lines and/or shaded regions.

4. The system (1) according to claim 1, wherein said user interface comprises a layout (3) of a plurality of graphical control elements (4, 5, 6, 7, 8), wherein at least one of said plurality of graphical control elements (4, 5, 6, 7, 8) is in the form of a dialogue box (13).

5. The system (1) according to claim 1, wherein said user interface comprises a plurality of graphical control elements (4, 5, 6, 7, 8), wherein a potentially imminent operator interaction with an individual graphical control element from said plurality of graphical control elements (4, 5, 6, 7, 8) is a measurable quantity that is measured in terms of either the proximity of an external element or the pressure applied by an external element, in the case operator interaction is undertaken by touchscreen, or in terms of the proximity of a mouse cursor element in the case operator interaction is undertaken by means of an external pointing device.

6. The system (1) according to claim 1, wherein said user interface comprises a plurality of graphical control elements (4, 5, 6, 7, 8), wherein a measured potentially imminent operator interaction with an individual graphical control element from said plurality of graphical control elements (4, 5, 6, 7, 8) activates a signal upon approaching a threshold value and deactivates said signal upon retreating from said threshold value, wherein said signal is in the form of a blue glowing effect and wherein said effect is spatially delimited to the immediate region of said individual graphical control element.

7. The system (1) according to claim 1, wherein said user interface comprises a plurality of graphical control elements (4, 5, 6, 7, 8) whereby the color of each of the plurality of graphical control elements (4, 5, 6, 7, 8) changes following an operator interaction, whereby the color of each of said plurality of graphical control elements (4, 5, 6, 7, 8) changes from black (10) to blue (12), in the event an associated function is activated and/or the color of each of said plurality of graphical control elements (4, 5, 6, 7, 8) changes from blue (12) to black (10), in the event an associated function is deactivated.

8. The system (1) according to claim 1, wherein said processor is configured to measure a potentially imminent operator interaction with an individual graphical control element from said plurality of graphical control elements (4, 5, 6, 7, 8) and to activate a signal upon approaching a threshold value and deactivate said signal upon retreating from said threshold value based on a measured potentially imminent operator interaction with an individual graphical control element from said plurality of graphical control elements (4, 5, 6, 7, 8).

9. The system (1) according to claim 1, wherein said processor is configured to change the color of each of the plurality of graphical control elements (4, 5, 6, 7, 8) following an operator interaction.

10. The system (1) according to claim 1, wherein said processor is configured to hide individual graphical control elements, from said plurality of graphical control elements, representing associated functions of heightened importance and/or criticality, from the remainder of a plurality of graphical control elements (4, 5, 6, 7, 8) such that accidental interaction with said hidden graphical control elements (4, 5, 6, 7, 8) is minimized, wherein access to said hidden graphical control elements (4, 5, 6, 7, 8) is achieved through operator interaction with a spatially separated and/or differently colored graphical control element (4, 8).

11. The system (1) according to claim 11, wherein said processor is configured to visualize at least one of said spatially separated graphical control elements (4, 8) translationally displaced by a distance exceeding a typical separation distance between the remainder of the graphical control elements.

12. The system (1) according to claim 1, wherein said processor is configured to accept an operator interaction within said dialogue box (13) only upon input of a preset minimum number of characters into said dialogue box (13).
